(19) United States
(12) Patent Application Publication
(10) Pub. No.: US 2010/0143985 A1
(43) Pub. Date: Jun. 10, 2010

(54) METHOD FOR PREPARING BUTANOL THROUGH BUTYRYL-COA AS AN INTERMEDIATE USING YEAST

(75) Inventors: Sang Yup Lee, Daejeon (KR); Eleftherios Terry Papoutsakis, Newark, DE (US); Yu-Sin Jang, Daejeon (KR)

Correspondence Address: MOORE & VAN ALLEN PLLC, P.O. BOX 13706, Research Triangle Park, NC, 27709, US

(73) Assignee: BIOFUELCHEM CO., LTD., Daejeon (KR)

(21) Appl. No.: 12/525,403

(22) PCT Filed: Feb. 11, 2008
(86) PCT No.: PCT/KR08/00787

§ 371 Date: Feb. 17, 2010

Related U.S. Application Data
(60) Provisional application No. 60/900,248, filed on Feb. 8, 2007.

(51) Int. Cl. C12P 17/18; C12N 1/19; C12P 7/16

(52) U.S. Cl. 435/119; 435/254.2; 435/160

(57) ABSTRACT

Disclosed herein are a method for producing butanol in yeast having the ability to biosynthesize butanol using butyryl-CoA as an intermediate, the method comprises producing butyryl-CoA in yeast having a CoAT (acetyl-CoA:butyryl-CoA CoA-transferase)-encoding gene introduced thereinto, through various pathways, and then converting the produced butyryl-CoA to butanol.

### FIELD OF THE INVENTION

The present invention relates to a method for producing butanol in yeast having the ability to biosynthesize butanol using butyryl-CoA as an intermediate.

### BACKGROUND ART

With the great increase in oil prices and growing concern about global warming and greenhouse gases, biofuels have recently gained increasing attention with respect to the production thereof using microorganisms. Particularly, biobutanol has an advantage over bioethanol in that it is more highly miscible with fossil fuels thanks to the low oxygen content thereof Recently, emerging as a substitute fuel for gasoline, biobutanol has been growing rapidly. The U.S. market for biobutanol amounts to 370 million gal per year, with a price of 3.75 $/gal. Butanol is superior to ethanol as a replacement for petroleum gasoline.

With high energy density, a low vapor pressure, a gasoline-like octane rating and low impurity content, it can be blended into existing gasoline at much higher proportions than ethanol without compromising performance, mileage, or organic pollution standards. The mass production of butanol by microorganisms can confer economic and environmental advantages of decreasing the import of crude oil and greenhouse gas emissions.

Butanol can be produced through anaerobic ABE (acetone-butanol-ethanol) fermentation by Clostridial strains (Jones, D. T. and Woods, D. R., Microbiol. Rev., 50:484, 1986; Rogers, P., Adv. Appl. Microbiol., 31:1, 1986; Lesnik, E. A. et al., Necleic Acids Research, 29: 3583, 2001). This biological method was the main technology for the production of butanol and acetone for more than 40 years, until the 1950s. Clostridial strains are difficult to improve further because of complicated growth conditions thereof and the insufficient provision of molecular biology tools and omics technology therefor.

Thus, it is suggested that microorganisms such as yeast, which has an excellent ability to produce ethanol and can be manipulated using various omics technologies, be developed as butanol-producing strains. Particularly, yeast to which little metabolic engineering and omics technology have been applied for the development of butanol-producing strains, have vast potential for development into butanol-producing strains.

Clostridium acetobutylicum produces butanol through the butanol biosynthesis pathway shown in FIG. 1 (Jones, D. T. and Woods, D. R., Microbiol. Rev., 50:484, 1986; Desai, R. P. et al., J. Biotechnol., 71:191, 1999). Two typical strains, Clostridium sp. and E. coli, which have been studied for the production of biobutanol, are difficult to use in industrial applications due to their tolerance to the final product, butanol. Meanwhile, recombinant bacteria capable of producing butanol, into which a butanol biosynthesis pathway is introduced, and butanol production using the same have been disclosed (US 2007/0259410 A1; US 2007/0259411 A1), but the production efficiency was modest.

Currently, yeasts are frequently used in the ethanol fermentation industry, and have a significantly high tolerance to alcohol. Generally, these yeasts have high metabolic activity and high growth rate, and grow well in an environment having low pH, low temperature and low water activity, like mold, and also mostly grow even in anaerobic conditions. Such properties are expected to provide the greatest advantages in producing butanol using yeasts. However, as shown in FIG. 2, yeasts cannot naturally produce butanol in general conditions. Also, there has been an attempt to produce butanol using recombinant yeasts, but the production of butanol was insignificant (WO 2007/041269 A2).

Accordingly, the present inventors have made many efforts to develop a novel method for producing butanol using yeast and, as a result, have found that an intermediate butyryl-CoA, produced in yeast using various pathways, is converted to butanol by the action of alcohol/aldehyde dehydrogenase (AAD), thereby completing the present invention.

### SUMMARY OF THE INVENTION

Therefore, it is an object of the present invention to provide a method for producing butanol, the method comprising producing butyryl-CoA, which is an important intermediate in a butanol-biosynthesizing pathway in yeast, through various pathways, and then producing butanol using the produced butyryl-CoA as an intermediate, as well as a recombinant yeast having the ability to biosynthesize butanol.

In order to accomplish the above object, the present invention provides a recombinant yeast having butanol-producing ability, into which a CoAT (CoA-transferase)-encoding gene capable of converting organic acid to organic acid-CoA by transferring a CoA moiety to organic acid, is introduced; and provides a method for producing butyryl-CoA and butanol, the method comprising culturing said recombinant yeast in a butyrate-containing medium.

The present invention also provides a method for producing butanol, the method comprising the steps of: co-culturing said recombinant yeast with a microorganism having butyrate-producing ability, such that butyrate is produced by the microorganiasm having butyrate-producing ability; allowing the recombinant yeast to produce butanol using the produced butyrate; and recovering butanol from the culture broth.

The present invention also provides a method for producing butyryl-CoA and butanol, the method comprises culturing yeast capable of biosynthesizing butyryl-CoA from fatty acids in a fatty acid-containing medium.

In the present invention, said yeast preferably has a gene encoding an AAD (alcohol/aldehyde dehydrogenase), which is expressed by itself to have AAD activity, or is introduced with an AAD-encoding gene.

Other features and aspects of the present invention will be apparent from the following detailed description and the appended claims.

### BRIEF DESCRIPTION OF DRAWINGS

FIG. 1 shows the butanol-producing pathway in Clostridium acetobutylicum.

FIG. 2 shows a part of the butanoate metabolic pathway in yeast. In FIG. 2, the dotted line indicates pathways not present in yeast, and the solid line indicates pathways present in yeast.

FIG. 3 shows a predicted pathway producing butanol using the butyryl-CoA pool in a recombinant yeast, from fatty acids.

FIG. 4 shows a pathway which produces butanol in a recombinant yeast according to the present invention by increasing the acetyl-CoA pool in the yeast cells using butyrate or acetate in a medium.

FIG. 5 shows a genetic map of a pYUC18 vector.

FIG. 6 shows a genetic map of pYUC18.adhE1.

FIG. 7 shows a genetic map of pYUC18.adhE1.ctfAB.

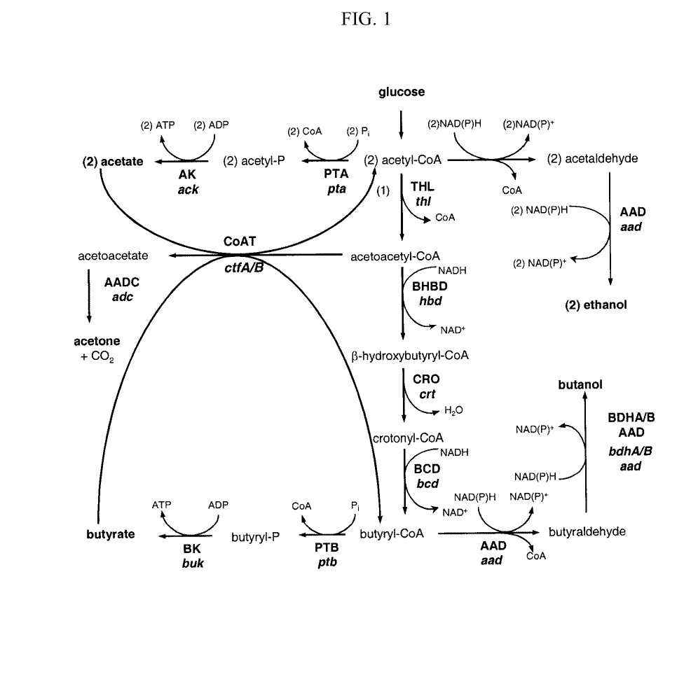

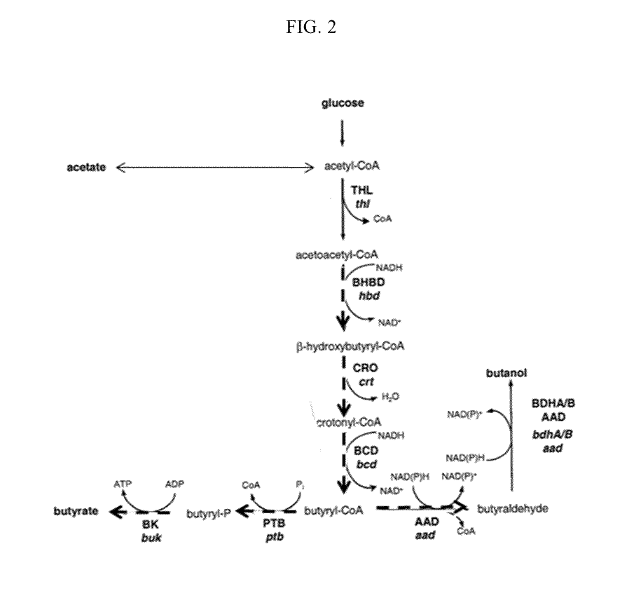

### DETAILED DESCRIPTION OF THE INVENTION, AND PREFERRED EMBODIMENTS

In the present invention, two methods were studied to produce butyryl-CoA in yeast: (1) a method for producing butyryl-CoA by introducing a CoAT (CoA transferase)-encoding gene into a yeast having a THL (an enzyme converting acetyl-CoA to acetoacetyl-CoA)-encoding gene so as to construct a recombinant yeast, and culturing the recombinant yeast in a butyrate-containing medium; and (2) a method for producing butyryl-CoA from fatty acids using the beta-oxidation pathway in yeast itself.

Yeast can produce short chain length (scl) and medium chain length (mcl) acyl-CoAs in peroxisome and cytosol by the beta-oxidation pathway using various fatty acids (Leaf, T. A. et al., Microbiology-Uk, 142:1169, 1996; Carlson, R. et al., J. Biotechnol., 124:561, 2006; Zhang, B. et al., Appl. Environ. Microbiol., 72:536, 2006), but there is no report yet on the production of butanol using the same.

The present inventors attempted to construct a recombinant yeast having an AAD (alcohol/aldehyde dehydrogenase)-encoding gene (adhE1) derived from Clostridium acetobutylicum ATCC 824 introduced thereinto to produce butanol from an intermediate butyryl-CoA expected to be produced by said two methods. In addition, the present inventors studied whether butanol is produced even when yeast without Clostridial AAD activity is cultured in fatty acid-containing medium.

As a result, it was confirmed that: (1) when a recombinant yeast, obtained by introducing a CoAT-encoding gene and an AAD-encoding gene into yeast having a THL-encoding gene, was cultured in a butyrate-containing medium, butanol was produced; (2) even when a recombinant yeast having an AAD-encoding gene introduced thereinto was cultured in a fatty acid-containing medium, butanol was also produced; and (3) when the yeast without Clostridial AAD activity is cultured in a fatty acid-containing medium, butanol was produced. Such results suggest that butyryl-CoA, produced from fatty acids by the beta-oxidation pathway, was converted to butanol by AAD which was expressed by itself. This indirectly indicates that the yeast, used in the present invention, has a gene which is expressed by itself to have AAD activity.

From the above results, it can be seen that the yeast having a gene which is expressed by itself to have AAD activity, can be used to produce butanol from butyryl-CoA synthesized through various pathways. Alternatively, when the yeast having no AAD activity therein is used, the recombinant yeast having the AAD-encoding gene introduced thereinto (e.g., Clostridium acetobutylicum ATCC 824-derived adhE1), can be used to produce butanol from butyryl-CoA.

Accordingly, in one aspect, the present invention relates to a recombinant yeast having butanol-producing ability, into which a CoAT (CoA-transferase)-encoding gene capable of converting organic acid to organic acid-CoA by transferring a CoA moiety to organic acid, is introduced; and to a method for producing butyryl-CoA and butanol, the method comprising culturing said recombinant yeast in a butyrate-containing medium.

In the present invention, said yeast preferably has a gene encoding an enzyme (THL) converting acetyl-CoA to acetoacetyl-CoA, said CoAT is preferably acetyl-CoA:butyryl-CoA CoA-transferase, and said CoAT-encoding gene is preferably Clostridium sp.-derived ctfAB, but the scope of the present invention is not limited thereto.

In another aspect, the present invention relates to a method for producing butyryl-CoA and butanol, which comprises culturing yeast capable of biosynthesizing butyryl-CoA from fatty acids in a fatty acid-containing medium.

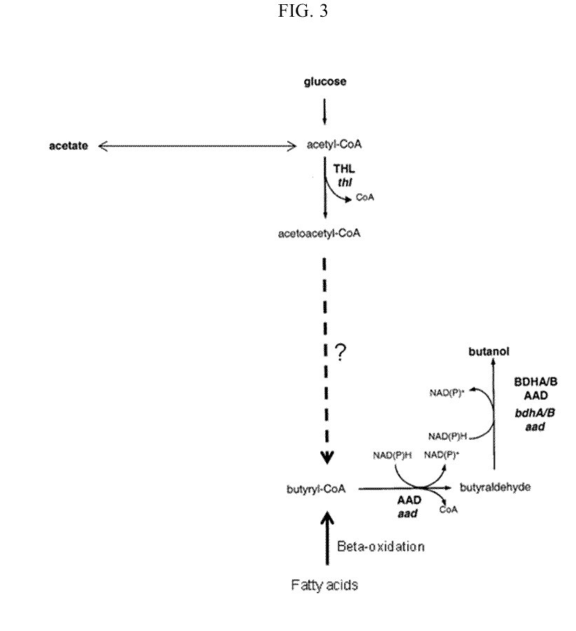

In one example of the present invention, the butanol-producing ability of a recombinant yeast [S. cerevisea (pYUC18.adhE1)] having an AAD (alcohol/aldehyde dehydrogenase)-encoding gene (adhE1) derived from Clostridium acetobutylicum ATCC 824 introduced thereinto, was analyzed in order to examine whether the recombinant yeast would produce an intermediate butyryl-CoA from acetyl-CoA or short-, medium- or long-chain fatty acids by the enzymes present in the yeast itself. The recombinant yeast was constructed in order to produce butanol from butyryl-CoA produced in the yeast itself via butyraldehyde. Specifically, it was predicted that, when various acyl-CoAs (butyryl-CoA, acetyl-CoA, etc.) are used in the recombinant yeast, the production of butanol would become possible. Furthermore, it was predicted that butanol would be produced from butyryl-CoA by AAD (alcohol/aldehyde dehydrogenase), introduced into or present in the recombinant yeast (FIG. 3).

In order to confirm this prediction, the recombinant yeast was cultured in an oleic acid/lauric acid-containing SC-dropout medium. As a result, it could be observed that butanol was produced from acyl-CoA, including butyryl-CoA, synthesized from the beta-oxidation pathway. Also, it was observed that butanol was also produced in a strain without Clostridial AAD activity. This is believed to be attributable to enzymes involved in the synthesis of acyl-CoA, which are present in the recombinant yeast and yeast itself having AAD activity. Specifically, it can be predicted that the reason why butanol is produced by culturing the recombinant yeast [S. cerevisea (pYUC18 adhE1)] and yeast itself having AAD activity, in the fatty acid-containing medium, is because fatty acid is converted to scl-acyl-CoA or mcl-acyl-CoA, such as butyryl-CoA, by the action of the enzymes (acyl-CoA synthases) (FIG. 3). Thus, it could be confirmed in the present invention that the enzymes (acyl-CoA synthases) present in the yeast, which convert fatty acids to scl-acyl-CoA or mcl-acyl-CoA, such as butyryl-CoA, contribute to the production of butanol (Marchesini, S. et al., J. Biol. Chem. 278:32596, 2003; Zhang, B. et al., Appl. Environ. Microbiol. 72:536, 2006).

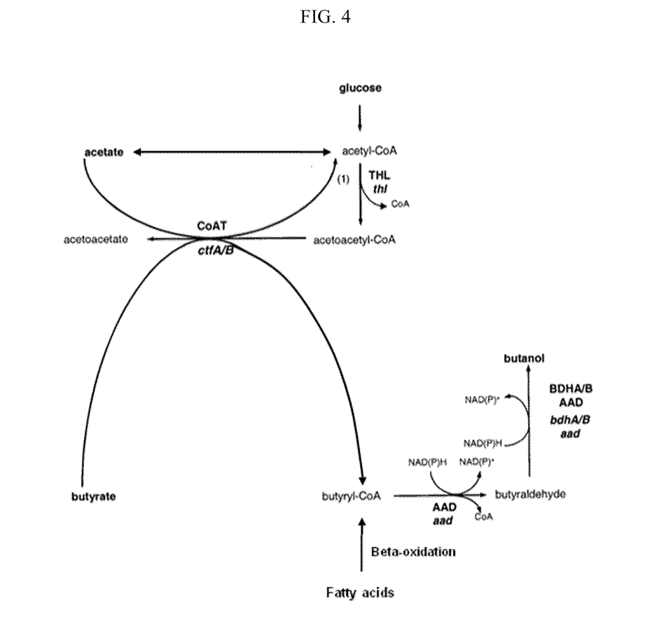

In another example of the present invention, experiments were carried out to examine whether the recombinant yeast having an alcohol/aldehyde dehydrogenase (AAD)-encoding gene (adhE1) and a CoA transferase (CoAT)-encoding gene, derived from Clostridium acetobutylicum ATCC 824 introduced thereinto, can increase the butyryl-CoA pool in the cells using butyrate of external origin to synthesize butanol using the same. The recombinant yeast [S. cerevisea (pYUC18.adhE1.ctfAB)] was constructed in order to produce butyryl-CoA using external butyrate and produce butanol from the butyryl-CoA via butyraldehyde. Clostridium acetobutylicum ATCC 824-derived CoAT enzyme is highly advantageous for increasing the butyryl-CoA pool in the yeast cells, because it transfers the CoA moiety of acetoacetyl-CoA to butyryl-CoA or acetyl-CoA (FIG. 4) (Bermejo, L. et al., Appl. Environ. Microbiol., 64:1079, 1998). Specifically, it was predicted that, when the recombinant yeast having an AAD-encoding gene (adhE1) and a CoAT-encoding gene (ctfAB) introduced thereinto, is cultured in a butyrate-containing medium, the butyryl-CoA pool in the yeast cells can be increased, thus increasing the production of butanol. Also, it was predicted that, when the recombinant yeast is cultured in a medium containing both butyrate and fatty acid, the butyryl-CoA pool in the yeast cells can be further increased, thus further increasing the production of butanol (FIG. 4).

To confirm this presumption, the recombinant yeast [S. cerevisea (pYUC18.adhE1.ctfAB)] was cultured in a butyrate-containing medium and, as a result, it could be observed that butanol was produced from butyrate via butyryl-CoA. This is believed to be attributable to the CoAT enzyme present in the recombinant yeast which is involved in the production of butyryl-CoA. It could be confirmed in the present invention that CoAT present in the recombinant yeast, which convert butyrate or acetate to butyl-CoA or acetyl-CoA, contributed to the production of butanol. In addition, it was observed that, when the recombinant yeast was cultured in the medium containing both butyrate and fatty acid, the production of butanol was further increased. This suggests that much more butyryl-CoA was biosynthesized from butyrate and fatty acid through the CoAT enzymes and the beta-oxidation pathway.

In the present invention, the fatty acid preferably has 4-24 carbon atoms and contains at least one selected from the group consisting of oleic acid and lauric acid.

In the present invention, the AAD- and CoAT-encoding genes are Clostridium sp.-derived adhE1 and ctfAB, respectively, but the scope of the present invention is not limited thereto. For example, genes derived from other microorganisms can be used without limitation in the present invention, as long as they can be introduced and expressed in the host yeast to show the same enzymatic activities as those of the above-described genes.

Meanwhile, in addition to the method of adding external butyrate directly to the recombinant yeast, a co-culture method may also be used to provide butyrate. Specifically, a strain capable of producing butyrate may be co-cultured with the recombinant yeast of the present invention, such that the precursor butyrate can be produced by the butyrate-producing strain, and the produced butyrate can be converted to butanol via butyryl-CoA by the present recombinant yeast.

Examples of co-culturing strain to produce specific products via precursors include Ruminococcus albus and Wolinella succinogenes. The fermentation of glucose through the pure culture of R. albus produces CO₂, H₂ and ethanol as final products in addition to the main product acetic acid. However, when R. albus is co-cultured with W. succinogenes, hydrogen is removed, and thus ethanol is not produced. Herein, W. succinogenes can produce acetate from acetyl-CoA to form ATP, and thus the production yield of ATP per mole of glucose can be increased compared to the case of R. albus. Specifically, co-culture with W. succinogenes is more effective in producing the final product acetic acid through the supply of required ATP, compared to the pure culture of R. albus (Stams, A. J., Antonie Van Leeuwenhoek, 66:271, 1994).

Microorganisms capable of producing butyrate include Clostridium sp. microorganisms (Clostridium butyricum, Clostridium beijerinckii, Clostridium acetobutylicum, etc.) and intestinal microorganisms (Megasphaera elsdenii, Mitsuokella multiacida, etc.) (Alam, S. et al., J. Ind. Microbiol., 2:359, 1988; Andel, J. G. et al., Appl. Microbiol. Biotechnol., 23:21-26, 1985; Barbeau, J. Y. et al., Appl. Microbiol. Biotechnol., 29:447, 1988; Takamitsu, T. et al., J. Nutr., 132:2229, 2002). When the butyrate-producing strain is co-cultured with the recombinant yeast of the present invention, butyrate will be produced by the strain, and the recombinant yeast of the present invention can produce butanol using the produced butyrate.

Accordingly, in another aspect, the present invention relates to a method for producing butanol, the method comprising the steps of: co-culturing said recombinant yeast with a microorganism having butyrate-producing ability, such that butyrate is produced by the microorganiasm having butyrate-producing ability; allowing the recombinant yeast to produce butanol using the produced butyrate; and recovering butanol from the culture broth.

Although only Clostridium sp. microorganisms and intestinal microorganisms have been mentioned as the butyrate-producing strain that may be used in the co-culture, it will be obvious to those skilled in the art that any strain may be used without limitation in the present invention, as long as it can produce butyrate and can be co-cultured with the recombinant yeast.

### Examples

Hereinafter, the present invention will be described in further detail with reference to examples. It is to be understood, however, that these examples are illustrative only, and the scope of the present invention is not limited thereto.

Particularly, although the following examples illustrated only S. cerevisea as yeast, the use of other yeasts will also be obvious to those skilled in the art. In addition, although the following examples illustrated only a specific strain-derived gene as a gene to be introduced, those skilled in the art will appreciate that any gene can be used as a gene to be introduced, as long as it is expressed in a host cell to show the same activity as that of the above gene.

Also, it should be noted that although only specific culture media and methods are exemplified in the following example, saccharified liquid, such as whey, CSL (corn steep liquor), etc, and the other media, and various culture methods, such as fed-batch culture, continuous culture, etc. (Lee et al., Bioprocess Biosyst. Eng., 26:63, 2003; Lee et al., Appl. Microbiol. Biotechnol., 58:663, 2002; Lee et al., Biotechnol. Lett., 25:111, 2003; Lee et al., Appl. Microbiol. Biotechnol., 54:23, 2000; Lee et al., Biotechnol. Bioeng., 72:41, 2001) also fall within the scope of the present invention.

### Example 1

### Preparation of Recombinant DNA Having Pathway Producing Butanol from Butyryl-CoA Introduced Thereinto

C. acetobutylicum ATCC 824 adhE1 (AAD-encoding gene), which is a gene in the final step of butanol biosynthesis pathway, was amplified and cloned into a pYUC18 expression vector, thus obtaining a pYUC18.adhE1 vector.

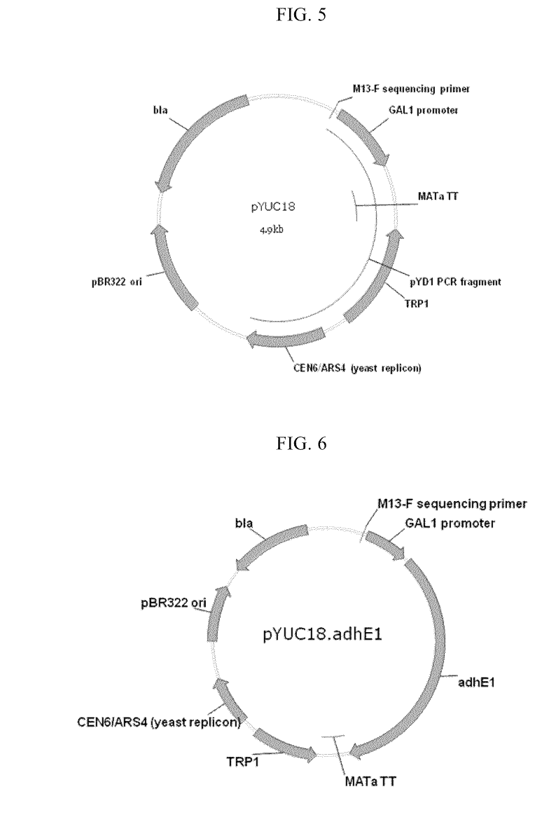

The expression vector pYUC18 was constructed by inserting a replication origin, a promoter, a transcription termination sequence, which have activity in yeast, into the E. coli cloning vector pUC18 (Amersham) as a backbone. pYD1 (Invitrogen) as a template was amplified by PCR using primers of SEQ ID NOs: 1 and 2 for 30 cycles of denaturation at 95° C. for 20 sec, annealing at 55° C. for 30 sec and extension at 72° C. for 30 sec, thus obtaining a PCR fragment (GAL promoter). Also, a PCR reaction was performed using primers of SEQ ID NOs: 3 and 4 in the same manner as described above, thus obtaining a PCR fragment (transcription termination sequence, TRP1 ORF, replicon). Then, the first PCR fragment and the second PCR fragment as templates were simultaneously subjected to PCR using primers of SEQ ID NOs: 1 and 4, thus obtaining a final PCR fragment in which the first and second PCR fragments were linked with each other. The amplified PCR fragment was digested with HindIII-SacI, and cloned into the pUC18 vector digested with the same enzyme (HindIII-SacI), thus constructing yeast expression vector pYUC18 (FIG. 5).

P1:

[SEQ ID NO: 1]

5′-aaaaaagcttaacaaaagctggctagtacgg-3′
P2:

[SEQ ID NO: 2]

5′-ggtacccggggatccgtcgacctgcagtccctatagtgagtcgtatt
acagc-3′
P3:

[SEQ ID NO: 5]

5′-ctgcaggtcgacggatccccgggtacccagtgtagatgtaacaaaat
cgact-3′
P4:

[SEQ ID NO: 4]

5′-ctaggagctcctgggtccttttcatcacgt-3′

The chromosomal DNA of Clostridium acetobutylicum ATCC 824 as a template was amplified by PCR using primers of SEQ ID NOs: 5 and 6, thus obtaining a PCR fragment. The amplified PCR fragment (adhE1 gene) was digested with PstI-XmaI and cloned into the expression vector pYUC18, thus constructing pYUC18.adhE1 (FIG. 6).

[SEQ ID NO: 5]

P5: 5′-aaaactgcagaagtgtatatttatgaaagtcacaacag-3′

[SEQ ID NO: 6]

P6: 5′-tccccccggggttgaaatatgaaggtttaaggttg-3′

### Example 2

### Preparation of Recombinant DNA Having AAD and CoAT Introduced Thereinto

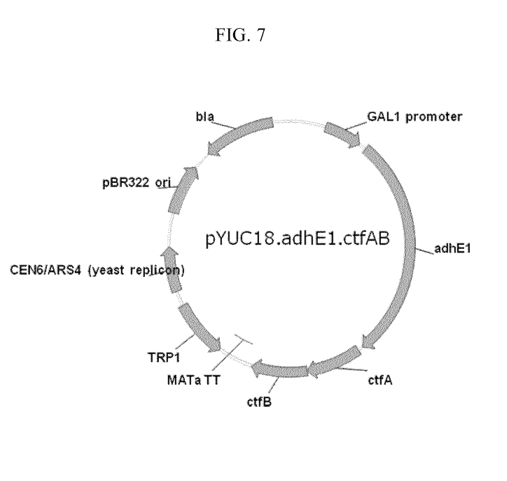

C. acetobutylicum ATCC 824 adhE1 (AAD-encoding gene) and ctfAB (CoAT-encoding gene) were amplified and cloned into the pYUC18 expression vector constructed in Example 1, thus obtaining a pYUC18.adhE1.ctfAB vector (FIG. 7).

The chromosomal DNA of Clostridium acetobutylicum ATCC 824 as a template was amplified by PCR using primers of SEQ ID NOs: 7 and 8, thus obtaining a PCR fragment. The amplified PCR fragment (adhE1-ctfAB gene) was digested with SalI-XmaI and cloned into the pYUC18 expression vector digested with the same enzyme, thus constructing pYUC18.adhE1.ctfAB (FIG. 7).

P7:

[SEQ ID NO: 7]

5′-tacgcgtcgacaagtgtatatttatgaaagtcacaacag-3′
P8:

[SEQ ID NO: 8]

5′-tccccccgggataccggcatgcagtatttctttctaaacagccat
g-3′

### Example 3

### Preparation of Recombinant Yeast Having AAD and/or CoAT Introduced Thereinto

Each of pYUC18, pYUC18.adhE1 and pYUC18.adhE1.ctfAB, prepared in Examples 1 and 2, was introduced into the S. cerevisea ATCC 208289 strain and colonies were screened in a SC-Trp selection medium (Bacto-yeast nitrogen base without amino acids (0.67%, Difco), glucose (2%, CJ), dropout mixture (0.2%, TRP DO supplement, BD Bioscience), Bacto-agar (2%, Difco)), thus constructing S. cerevisea (pYUC18), S. cerevisea (pYUC18.adhE1) and S. cerevisea (pYUC18.adhE1 ctfAB) strains.

### Example 4

### Production of Butanol in Yeast by Addition of Fatty Acid

The production of butanol was attempted by culturing the recombinant yeast S. cerevisea (pYUC18.adhE1), constructed in Example 3. The basic composition of a medium used in the culture was as follows: Bacto-yeast nitrogen base without amino acids (0.67%, Difco), glucose (2%, CJ), uracil (20 mg/l, Sigma), L-leucin (100 mg/l, Sigma), and L-histidine (20 mg/l, Sigma). Also, the basal medium was supplemented with 2.5 g/l of oleic acid and 2.5 g/l of lauric acid and adjusted to a pH of 5.7.

100 ml of the medium was added to a 250 ml culture flask, and the recombinant yeast S. cerevisea (pYUC18 adhE1) was inoculated into the medium and cultured in aerobic and anaerobic chambers at 30° C. After the culture process, samples were collected from the culture at 12-hr intervals, and butanol in the sample was quantified by Gas-chromatography (GC, Agillent).

As a result, as shown in Table 1 below, it could be observed that butanol was produced not only in the S. cerevisea (pYUC18.adhE1) strain, but also in the S. cerevisea (pYUC18) strain. This suggests that the fatty acid added to the medium was converted to various acyl-CoA pools, including butyryl-CoA, by beta-oxidation, and then converted to butanol.

TABLE 1
Butanol concentration (mg/l) of supernatants from cultures of
S. cerevisea strains challenged with fatty acids (5 g/l)

| | S. cerevisea | S. cerevisea |
|---|---|---|
| Culture condition | (pYUC18) | (pYUC18.adhE1) |
| aerobic | 0.5 | 0.2 |
| anaerobic | 1.8 | 1.8 |

### Example 5

### Production of Butanol in Recombinant Yeast by Addition of Butyrate

The production of butanol was attempted by culturing the recombinant yeast S. cerevisea (pYUC18.adhE1.ctfAB), constructed in Example 3. The composition of a basal medium used in the culture was the same as that used in Example 4. Also, the basal medium was supplemented with 40 mM butyric acid and adjusted to a pH of 5.7.

100 ml of the medium was added to a 250 ml culture flask, and the recombinant yeast S. cerevisea (pYUC18.adhE1.ctfAB) was inoculated into the medium and cultured in aerobic and anaerobic chambers at 30° C. After the culture process, samples were collected from the culture at 12-hr intervals, and butanol in the sample was quantified by Gas-chromatography (GC, Agillent).

As a result, as shown in Table 2 below, the production of butanol was not observed in the yeast S. cerevisea (pYUC18), whereas butanol was produced in the recombinant yeast S. cerevisea (pYUC18.adhE1.ctfAB). This suggests that, when the strain having CoAT introduced thereinto is cultured in the medium supplemented with butyrate, the butyryl-CoA pool in the recombinant cells increases, and thus butanol is produced by the recombinant cells.

TABLE 2
Butanol concentration (mg/l) of supernatants from cultures of yeast
challenged with butyric acid (40 mM)

| Culture | S. cerevisea | S. cerevisea |
|---|---|---|
| condition | (pYUC18) | (pYUC18.adhE1.ctfAB) |
| aerobic | 0 | 0.5 |
| anaerobic | 0 | 1.2 |

Also, the butyrate-supplemented medium was additionally supplemented with fatty acid, and each of the yeasts was cultured in the medium. Then, butanol in the samples collected from the cultures was quantified. As a result, as shown in Table 3 below, butanol was also produced in the case where the recombinant yeast was cultured in the butyrate-supplemented medium additionally supplemented with fatty acid. Also, it could be observed that the recombinant strain S. cerevisea (pYUC18.adhE1.ctfAB) produced butanol at a concentration higher than that in the S. cerevisea (pYUC18) strain. This suggests that the recombinant strain S. cerevisea (pYUC18.adhE1.ctfAB), which has both (1) the metabolic pathway converting fatty acid to butyryl-CoA by the action of acyl-CoA synthase and (2) the metabolic pathway converting butyrate to butyryl-CoA through the action of CoAT, is more advantageous for butanol synthesis. Also, it can be seen that the metabolic pathway biosynthesizing butyryl-CoA as an intermediate, plays an important role in the production of butanol.

TABLE 3
Butanol concentration (mg/l) of supernatants from cultures of yeast
challenged with butyric acid (20 mM) and fatty acids (5 g/l)

| Culture | S. cerevisea | S. cerevisea |
|---|---|---|
| condition | (pYUC18) | (pYUC18.adhE1.ctfAB) |
| aerobic | 0.6 | 0.8 |
| anaerobic | 1.5 | 2.8 |

### INDUSTRIAL APPLICABILITY

As described in detail above, the present invention has an effect to provide a method for producing butanol in yeast, the method comprising producing butyryl-CoA in yeast using various pathways, and then producing butanol using the produced butyryl-CoA as an intermediate.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

1. A recombinant yeast having butanol-producing ability, into which a CoAT (CoA-transferase)-encoding gene capable of converting organic acid to organic acid-CoA by transferring a CoA moiety to organic acid, is introduced.

2. The recombinant yeast having butanol-producing ability according to claim 1, wherein said CoAT is acetyl-CoA:butyryl-CoA CoA-transferase.

3. The recombinant yeast having butanol-producing ability according to claim 2, wherein said CoAT-encoding gene is Clostridium sp.-derived ctfAB.

4. The recombinant yeast having butanol-producing ability according to claim 1, wherein said yeast has a gene encoding an enzyme (THL) converting acetyl-CoA to acetoacetyl-CoA.

5. A method for producing butyryl-CoA, the method comprises culturing the recombinant yeast of claim 1, in a butyrate-containing medium.

6. The method for producing butyryl-CoA according to claim 5, wherein said medium further contains fatty acid.

7. The method for producing butyryl-CoA according to claim 6, wherein the fatty acid has 4-24 carbon atoms.

8. A method for producing butanol, the method comprises culturing the recombinant yeast of claim 1, in a butyrate-containing medium to produce butanol; and recovering the produced butanol from the culture broth.

9. The method for producing butanol according to claim 8, wherein said yeast is expressed by itself to have a gene encoding an AAD (alcohol/aldehyde dehydrogenase), showing AAD activity.

10. The method for producing butanol according to claim 8, wherein said yeast is a recombinant yeast having the AAD-encoding gene introduced thereinto.

11. The method for producing butanol according to claim 10, wherein the AAD-encoding gene is adhE1 or adhE2 derived from Clostridium sp.

12. The method for producing butanol according to claim 8, wherein said medium further contains fatty acid.

13. The method for producing butanol according to claim 12, wherein the fatty acid has 4-24 carbon atoms.

14. A method for producing butanol, the method comprising the steps of:
co-culturing the recombinant yeast of claim 1 with a microorganism having butyrate-producing ability, such that butyrate is produced by the microorganiasm having butyrate-producing ability; allowing the recombinant yeast to produce butanol using the produced butyrate; and
recovering butanol from the culture broth.

15. The method for producing butanol according to claim 14, wherein said yeast is expressed by itself to have a gene encoding an AAD (alcohol/aldehyde dehydrogenase), showing AAD activity.

16. The method for producing butanol according to claim 14, wherein said yeast is a recombinant having the AAD-encoding gene introduced thereinto.

17. The method for producing butanol according to claim 16, wherein the AAD-encoding gene is adhE1 or adhE2 derived from Clostridium sp.

18. The method for producing butanol according to claim 14, wherein said medium further contains fatty acid.

19. The method for producing butanol according to claim 18, wherein the fatty acid has 4-24 carbon atoms.

20. A method for producing butyryl-CoA, the method comprises culturing yeast capable of biosynthesizing butyryl-CoA from fatty acids in a fatty acid-containing medium.

21. The method for producing butyryl-CoA according to claim 20, wherein the fatty acid has 4-24 carbon atoms.

22. A method for producing butanol, the method comprises:
culturing yeast capable of biosynthesizing butyryl-CoA from fatty acids in a fatty acid-containing medium to produce butanol; and
recovering the produced butanol from the culture broth.

23. The method for producing butanol according to claim 22, wherein the fatty acid has 4-24 carbon atoms.

24. The method for producing butanol according to claim 22, wherein said yeast is expressed by itself to have a gene encoding an AAD (alcohol/aldehyde dehydrogenase), showing AAD activity.

25. The method for producing butanol according to claim 24, wherein said yeast is a recombinant yeast having the AAD-encoding gene introduced thereinto.

26. The method for producing butanol according to claim 25, wherein the AAD-encoding gene is adhE1 or adhE2 derived from Clostridium sp.


SEQUENCE LISTING (8 sequences)

SEQ ID NO: 1 - DNA, 31 bp, Artificial Sequence
  p1 primer
aaaaaagcttaacaaaagctggctagtacgg

SEQ ID NO: 2 - DNA, 52 bp, Artificial Sequence
  p2 primer
ggtacccggggatccgtcgacctgcagtccctatagtgagtcgtattacagc

SEQ ID NO: 3 - DNA, 52 bp, Artificial Sequence
  p3 primer
ctgcaggtcgacggatccccgggtacccagtgtagatgtaacaaaatcgact

SEQ ID NO: 4 - DNA, 30 bp, Artificial Sequence
  p4 primer
ctaggagctcctgggtccttttcatcacgt

SEQ ID NO: 5 - DNA, 38 bp, Artificial Sequence
  p5 primer
aaaactgcagaagtgtatatttatgaaagtcacaacag

SEQ ID NO: 6 - DNA, 35 bp, Artificial Sequence
  p6 primer
tccccccggggttgaaatatgaaggtttaaggttg

SEQ ID NO: 7 - DNA, 39 bp, Artificial Sequence
  p7 primer
tacgcgtcgacaagtgtatatttatgaaagtcacaacag

SEQ ID NO: 8 - DNA, 46 bp, Artificial Sequence
  p8 primer
tccccccgggataccggcatgcagtatttctttctaaacagccatg